(19) United States
(12) Patent Application Publication
(10) Pub. No.: US 2016/0263172 A1
(43) Pub. Date: Sep. 15, 2016

(54) USES OF CAMPHOR OIL SOLUTION

(71) Applicant: CHING-CHUAN WU, Kaohsiung City (TW)

(72) Inventor: CHING-CHUAN WU, Kaohsiung City (TW)

(21) Appl. No.: 14/657,388

(22) Filed: Mar. 13, 2015

(51) Int. Cl. A61K 36/54; A61K 47/10

(52) U.S. Cl. CPC A61K 36/54; A61K 47/10

(57) ABSTRACT

The invention provides a composition for treating a microbial disease in a bee. The composition comprises camphor oil and a volatile solvent. The invention also provides a method for treating a microbial disease in a bee comprising administrating the bee with a camphor oil solution, wherein the camphor oil solution comprises camphor oil and a volatile solvent, and the volatile solvent is able to dissolve the camphor oil.

### BACKGROUND OF THE INVENTION

1. Field of the Invention

The invention relates to a veterinary drug. More particularly, the invention provides a composition for treating a microbial disease in a bee. 2. Description of the Related Art

Cultivation of bees that are one of important insects in agriculture has been developed for thousands of years. On the other hand, bees also play the most critical role in biological chain. Human breeds bees for acquiring products, such as honey, royal jelly, pollen, propolis, brood, beeswax, or venom. In nature, when bees harvest pollen and nectar, they also help plants pollinate at the same time for reproduction and fruit.

In cultivation process, bee nests, bee eggs, larvae and bees all suffer from microbial diseases. Because bees are bred by high density cultivation, if bees suffer from microbial diseases, the diseases gradually spread to the whole bee nest, beehive, apiary, and even the entire region. If bees cannot help pollinate, human cannot harvest some crops, and prices of many crops, such as apples, blueberries, cauliflowers, almonds, rise dramatically. Such phenomenon also leads many plants to extinct. Therefore, bees play an important role for providing human food. Hence, the microbial diseases of bees not only effect beekeepers but also influence overall ecology.

In general, common microbial diseases of bees include diseases caused by a fungus, a bacterium and a virus. For example, the diseases caused by a mold, a bacterium and yeast in every stage of bees including bee nests, bee surfaces, pollen balls and nectar stored in cocoons, bee surfaces and larvae bodies, are often found as mildew. Such microbes not only harm bees but also affect beekeepers' health due to contacting to initiate allergy symptoms. For example, pericytsdpis disease, also known as bee chalk brood disease, is a fungus infectious disease caused by pericytsdpis which attacks larvae and invades bee nests and bee cocoons through spores, and pericytsdpis disease is a common disease in cultivation of bees. For example, sac brood disease is a virus infectious disease for larvae, and the main infectious source is contaminated feed.

Currently, in accordance with the bee microbial disease, the prevention ways adopt the following methods: fumigating bee nests, bee cocoons by formaldehyde and sulfur; disinfecting by sodium hypochlorite, lime milk and alkaline solution; sterilizing by dry heat; disinfecting by sunlight; managing bees; adjusting microclimate of beehive and feeding non-contaminated feed or administrating antibiotics or anti-fungal agent. However, those conventional ways are completely unable to treat the microbial diseases. Contamination of bee products and ecological harm also happen caused by disinfection process or by medication overuse. The most effective way is still to disinfect and throw the infected nest pieces at infecting initial stage; nevertheless, this way leads beekeeper's economic damage.

### SUMMARY OF THE INVENTION

In the present invention, a secure, effective and easy to operate composition is provided to treat a microbial disease in a bee.

The present invention provides a composition for treating a microbial disease in a bee comprising:
camphor oil; and
a volatile solvent, and the volatile solvent is able to dissolve the camphor oil.

The present invention also provides a method for treating a microbial disease in a bee comprising administrating the bee with a camphor oil solution, wherein the camphor oil solution comprises camphor oil and a volatile solvent, and the volatile solvent is able to dissolve the camphor oil.

The present invention have been illustrated and described as following in detail. The technical features, purposes and advantages of the present invention have been shown in the following embodiments and the scope as defined in the following claims.

### DETAILED DESCRIPTION OF THE INVENTION

The present invention provides a composition for treating a microbial disease in a bee comprising:
camphor oil; and
a solvent comprises camphor oil and volatile solvent, and the volatile solvent is able to dissolve the camphor oil.

The present invention also provides a method for treating a microbial disease in a bee comprising administrating the bee with a camphor oil solution, wherein the solution is used to prepare drugs for treating bee microbial diseases and the camphor oil solution comprises a camphor oil and a solvent comprises camphor oil and volatile solvent, and the volatile solvent is able to dissolve the camphor oil.

The term “a bee” as used herein refers to a bee which can harvest pollen and nectar and brew honey. Preferably, the bee belongs to Animalia, Arthropoda, Insecta, Hymenoptera, Apocrita, Apoidea, and Apidae; more preferably, Apis mellifera. For example, the bee includes but not limited to Apis(Micrapis)andreniformis Smith, Apis florae Fabricius, Apis(Megapis)laboriosa Smith, Apis(Megapis)dorsata Fabricius, Apis(Sigmatapis)koschevnikovi Butte-Reepen, Apis(sigmatapis)muluensis Tingek, Koeniger and Koeniger, Apis(sigmatapis)nigrocinta Smith, Apis(sigmatapis)cerana Fabricius, or Apis mellifera Linnaeus.

The term “a microbial disease in a bee” as used herein refers to a disease caused by microbial infection in a bee. Preferably, a microbial pathogen of the microbial disease comprises a fungus, a bacterium and/or a virus. According to the invention, the fungus includes but not limited to Mucor spp., Aspergillosis (such as Aspergillus flavus, Aspergillus fumigatus), rhizopus, yeast, pericytsdpis, eurotium, entomophthoramycosis; according to the invention, the bacterium includes but not limited to bacilli (such as Bacillus larvae, Bacillus alvei, Bacillus hive, Septicemia bacilli), Escherichia coli, streptococcus (such as Streptococcus hive, Streptococcus bee); according to the invention, the virus includes but not limited to bee chronic paralysis virus, sac brood virus and bee pupa virus. Preferably, the microbial pathogen of the microbial disease comprises a mold and/or yeast; more preferably, the microbial pathogen of the microbial disease is Mucor spp.

The term “camphor oil” as used herein refers to oil ingredient extract from stems of a camphor tree, wherein the camphor tree includes but not limited to a camphor tree, a linaloe camphor tree, an oil camphor tree, and a stout camphor tree. The method to obtain camphor oil is by distillation, such as chipping the camphor tree, and distilling the chipping piece with high pressure. According to the invention, the camphor oil is colorless or yellowish to red brown and it belongs to high ash material.

According to the invention, preferably, the volatile solvent comprises an alcohol having 1 to 6 carbons or an ether having 1 to 6 carbons. The alcohol having 1 to 6 carbons includes but not limited to methanol, ethanol, propanol, isopropanol, butanol, second-butanol, tertiary butanol, amyl alcohol, isoamylalcohol, tert-amyl alcohol, neopentyl glycol, pentanediol, 1-hexanol, 2-hexanol, cyclohexanol, or hexanediol; the ether having 1 to 6 carbons includes but not limited to methyl ether, ethyl ether, N-propyl ether, isopropyl ether, dipropylether, N-butyl ether, second-ether, tertiary butyl ether, N-amyl ether, diisoamylether, tert-pentyl ether, neopentyl ether, glutaric ether, 1-hexylether, 2-hexylether, cyclohexyl ether, or adipic ether. Preferably, the volatile solvent is ethanol.

In one preferred embodiment of the invention, the volatile solvent is provided in a solution form. For example, the volatile solvent is an about 75% to 99.9% ethanol solution; preferably, is an about 90% to 99% ethanol solution; more preferably, is an about 95% ethanol solution.

The mixture ratio of the camphor oil and the volatile solvent can be adjusted depending on the demands; more preferably, the weight ratio of the camphor oil and the volatile solvent is from about 7:3 to about 1:1. If the weight ratio of the camphor oil and the volatile solvent is lower than about 1:1, bees are easy affected by the volatile solvent, and bees are “drunken”; if the weight ratio of the camphor oil and the volatile solvent is higher than about 7:3, the camphor oil solution has poor dissolution and volatilization.

Preferably, according to the invention, the camphor oil solution further comprises water for assisting adjustment of the dissolution of camphor oil, wherein the weight ratio of water in the camphor oil solution is from about 0.01% to about 1%; preferably, is from about 0.1% to about 0.5%; more preferably, is from about 0.2% to about 0.4%.

According to the invention, the composition is directly applied on bee nests which suffer from the microbial disease; preferably, the applied way includes but not limit to spray. The used amount and frequency depends on a degree of suffering from the microbial disease.

The following examples are given for the purpose of illustration only and are not intended to limit the scope of the present invention.

### A Composition for Treating a Microbial Disease in a Bee

Camphor oil, cypress oil, or peppermint and 95% ethanol solution are mixed to each other with the weight ratio of 6:4 to prepare a composition.

The composition is sprayed on the bee nests which are infected by Mucor spp. Only the composition comprising camphor oil is helpful which means that not all herbal oil having antibacterial and antifungal property to the microbial disease in a bee.

### Effect of Treatment of a Microbial Disease in a Bee

Camphor oil and 95% ethanol solution are mixed to each other with the weight ratio of 6:4 to prepare a composition. The composition is sprayed on the bee nests which are infected by mold and yeast, especially Mucor spp. The used amount and frequency depends on a degree of suffering from the microbial disease. Generally, 1000 c.c of composition can be used to spray 15 to 20 nest pieces, and an index is assayed depending on days of queen bees spawn on the infected region again. The results are shown in Table 1.

TABLE 1

| | | Days (queen bees spawn |
|---|---|---|
| | | on the infected region |
| nest piece condition | | again) |
| Treated with the | new nest piece less than 2 | 3 days |
| composition of the | years | |
| invention | old nest piece between 3 | 5 days |
| | to 5 years | |
| | nest piece with serious | 7 days |
| | infection | |
| | amount of mold and yeast | 1.6 × 10² CFU/g |
| Untreated with the | new nest piece less than 2 | 20 days |
| composition of the | years | |
| invention | old nest piece between 3 | 30 days |
| | to 5 years | |
| | nest piece with serious | 40 days or bees death |
| | infection | |
| | amount of mold and yeast | 4.9 × 10⁵ CFU/g |

Amounts of mold and yeast are measured by the detection method of Ministry of Health and Welfare 2003.05.27 no. 0929210167 food microbiology.

According to the results, the composition according to the invention can treat the microbial disease in the bee, and can recover the infected nest pieces, and shorten the days of infection to spawning.

While embodiments of the present invention have been illustrated and described, various modifications and improvements can be made by persons skilled in the art. It is intended that the present invention is not limited to the particular forms as illustrated, and that all modifications not departing from the spirit and scope of the present invention are within the scope as defined in the following claims.

What is claimed is:

1. A method for treating a microbial disease in a bee comprising administrating the bee with a camphor oil solution, wherein the camphor oil solution comprises camphor oil and a volatile solvent, and the volatile solvent is able to dissolve the camphor oil.

2. The method according to claim 1, wherein a microbial pathogen of the microbial disease comprises a fungus and/or a bacterium.

3. The method according to claim 1, wherein a microbial pathogen of the microbial disease comprises a mold and/or yeast.

4. The method according to claim 1, wherein a microbial pathogen of the microbial disease is Mucor spp.

5. The method according to claim 1, wherein the volatile solvent comprises an alcohol having 1 to 6 carbons or an ether having 1 to 6 carbons.

6. The method according to claim 1, wherein the volatile solvent is ethanol.

7. The method according to claim 1, wherein the weight ratio of the camphor oil and the volatile solvent is from about 7:3 to about 1:1.

8. The method according to claim 1, wherein the camphor oil solution further comprises water.

9. The method according to claim 8, wherein the weight ratio of water in the camphor oil solution is from about 0.01% to about 1%.

10. The method according to claim 1, wherein the weight ratio of water in the camphor oil solution is from about 0.1% to about 0.5%.
